(19) United States
(12) Patent Application Publication
(10) Pub. No.: US 2002/0132239 A1
(43) Pub. Date: Sep. 19, 2002

(54) Cell lineage markers

(76) Inventors: Robin Lovell-Badge, Mill Hill (GB); Larysa Halyna Pevny, Chapel Hill, NC (US); Vasso Episkopou, (US)

Correspondence Address: PALMER & DODGE, LLP, KATHLEEN M. WILLIAMS, 111 HUNTINGTON AVENUE, BOSTON, MA, 02199, US

(21) Appl. No.: 09/886,899

(22) Filed: Jun. 21, 2001

(30) Foreign Application Priority Data
Dec. 22, 1998 (GB) 9828383.1
Dec. 21, 1999 (GB) PCT/GB99/004336

(51) Int. Cl. C12Q 1/68; G01N 33/567; C12N 5/08

(52) U.S. Cl. 435/6; 435/7.21; 435/368

(57) ABSTRACT

Sox gene expression correlates in general with specific stages during embryogenesis. It has been determined that the expression of Sox genes may be used, as directed herein, to induce or select pluripotent cells which are at least partially committed to a given developmental pathway. There is provided a method for isolating a pluripotent cell which is at least partially committed to a given developmental pathway, comprising the steps of: a) selecting a population of pluripotent cells; b) sorting the cells according to Sox gene expression; and c) isolating those cells which express a give Sox gene.

### FIELD OF THE INVENTION

The present invention relates to a method of marking, selecting and generating committed or partially committed cell lineages from tissues. In particular, the invention relates to the use of the Sox genes for the selection or generation of various specified cell types.

### BACKGROUND OF THE INVENTION

SOX proteins constitute a family of transcription factors related to the mammalian testis determining factor SRY through homology within their HMG box DNA binding domains. In DNA binding studies, SOX proteins exhibit sequence specific binding; however, unlike most transcription factors, binding occurs in the minor groove resulting in the induction of a dramatic bend within the DNA helix. Although SOX proteins can induce transcription of reporter constructs in vitro and possess activation domains, transcriptional activation by these factors appears to be context dependent. In other words members of this family seem to act in conjunction with other proteins. Therefore, SOX proteins display properties of both classical transcription factors and architectural components of chromatin (reviewed by Pevny & Lovell-Badge, 1997).

Members of the Sox gene family are expressed in a variety of embryonic and adult tissues, where they appear to be responsible for the development and/or elaboration of particular cell lineages. Sry is transiently expressed in the precursor Sertoli cells of the XY genital ridge and is responsible for triggering development of the male phenotype (reviewed by Lovell-Badge & Hacker, 1995). Thus, the lack of Sry results in XY females and its gain in XX males. Sox9 is expressed in immature chondrocytes and male gonads, as well as certain other sites; mutations in the human SOX9 gene are associated with Campomelic Dysplasia, a human skeletal malformation syndrome, and XY female sex reversal. Sox4 is expressed in many tissues and a null mutation of the gene in mouse results in the absence of mature B cells and heart malformations. The Xsox17 gene is involved in endoderm formation in Xenopus embryos. The Xenopus SoxD gene mediates neural induction in frog embryos. Sox11 in mouse and human is involved in neural crest cell development, notably the enteric nervous system. These functional analyses suggest that Sox genes function in cell fate decisions in diverse developmental pathways.

A subfamily of Sox genes, that includes Sox1, Sox2 and Sox3, shows expression profiles during vertebrate embryogenesis that suggest the genes could function in the control of cell fate decisions within the early developing nervous system. Sox2 and Sox3 begin to be expressed at preimplantation and epiblast stages respectively, and are then restricted to the neuroepithelium. Sox1 appears only at approximately the stage of neural induction. Related to Sox1-3 are the chicken Sox14 and Sox2I, the zebrafish Sox19, the Xenopus SoxD and the Drosophila Sox70D (Dichaete), all of which are expressed at various stages during development in neural tissues. A number of other sox genes and their tissue distributions have been described (see table 1).

The molecular mechanisms controlling induction and determination of tissue development during embryogenesis have begun to be elucidated. The identification by cellular and biochemical methods, of secreted molecules involved in the development of cell fate illustrates the important role of the environment in specifying cell identity. In addition, a number of transcription factors have been isolated which play important roles in the specification and differentiation of neural cell lineages. For example, the characterization of vertebrate homologues of Drosophila proneural and neurogenic genes, which control neural specification in the fly, has revealed analogous molecular mechanisms in vertebrate neural cell fate determination and differentiation. Misexpression of these transcription factors involved in cell fate determination is observed to cause abnormalities in development.

In our co-pending international patent application PCT/GB98/01862, filed Jun. 25, 1998, we describe the use of the Sox1 gene and SOX1 polypeptide in inducing commitment to the neural pathway in pluripotent embryonal carcinoma cells, and in identifying cells committed to the neural fate.

### SUMMARY OF THE INVENTION

In accordance with the present invention, it has been found that Sox gene expression correlates in general with specific stages during embryogenesis. Moreover, it has been determined that the expression of Sox genes may be used, as described herein, to induce or select pluripotent cells which are at least partially committed to a given developmental pathway. According to a first aspect of the present invention, there is provided a method for isolating a pluripotent cell which is at least partially committed to a given developmental pathway, comprising the steps of:

(a) selecting a population of pluripotent cells;

(b) detecting Sox gene expression;

(c) sorting the cells according to Sox gene expression; and

(d) isolating those cells which express a given Sox gene.

As set forth in the following description: the Sox genes, which encode SOX proteins, are responsible for the specification of a variety of proliferating cells which are not yet totally committed, as well as acting as a marker for such cells. Expression of Sox genes is responsible for the generation of specific pluripotent cell lineages, which in vivo or in vitro are capable of differentiating into the many different cells which belong to a given developmental line.

As used herein, a “pluripotent cell” is a cell which may be induced to differentiate, in vivo or in vitro, into at least two different cell types. These cell types may themselves by pluripotent, and capable of differentiating in turn into further cell types, or they may be terminally differentiated, that is incapable of differentiating beyond their actual state. Pluripotent cells include totipotent cells, which are capable of differentiating along any chosen developmental pathway. For example, embryonal stem cells (Thomson et al., (1998) Science 282:1145-1147) are totipotent stem cells. Pluripotent cells also include other, tissue-specific stem cells, such as neuronal stem cells, neuroectodermal cells, ectodermal cells and endodermal cells, for example gut endodermal cells, and mesodermal stem cells, which have the ability to give muscle or skeletal components, dermal components such as skin or hair, blood cells, etc.

“Developmental pathway” refers to a common cell fate which can be traced from a particular precursor cell. Thus, for example, the neuronal developmental pathway defines the developmental changes that occur in those cells which develop from the neural plate and give rise to all the neural and glial cells and ganglia of an adult organism. They can alternatively be defined as cells of the “neural lineage”.

A “partially committed” cell is a cell type which is no longer totipotent but remains pluripotent. For example, neuroectodermal cells are capable of giving rise to any cell type in the CNS or PNS, yet are not able to give rise to endodermal tissues.

As used herein, “totipotent” refers to a cell that is capable of differentiating into any cell type or tissue of an organism.

Pluripotent cells may be “selected” by any one or more of a variety of means, including immunostaining or FACs analysis, and the term includes dissection of tissue types from developing embryos, isolation or generation of pluripotent, including totipotent, cells in vivo or in vitro. Preferably, the term refers to the isolation of one class of pluripotent cells from one or more other cell types. In the context of the present invention, this allows greater precision in selection using Sox genes because, as a result of their widespread expression, particular Sox genes cannot be generally stated to be exclusively associated with any one tissue. Thus, preselection of possible tissue types allows Sox gene expression to be used to accurately identify a desired cell lineage from a remaining cell population.

Cells can be sorted by affinity techniques, or by cell sorting (such as fluorescence-activated cell sorting, FACS) where they are labeled with a suitable label, such as a fluorophore conjugated to or part of, for example, an antisense nucleic acid molecule or an immunoglobulin, or an intrinsically fluorescent protein such as green fluorescent protein (GFP) or variants thereof. As used herein, “sorting” refers to the at least partial physical separation of a first cell type from a second.

“Isolating” cells refers to removing at least one component from a mixture in which the cells were previously associated. In the context of the present invention, “isolating” preferably refers to removal of at least one cell type from a mixed population of cells. Preferably, “isolating” can refer to the enrichment of a population of cells for a desired cell type. “Isolated” refers to a population of molecules or cells, the composition of which is less than 50%, preferably less than 40% and most preferably 2% or less, contaminating molecules or cells of an unlike nature. Preferably, “isolating” refers to substantial purification such that there is only a single cell type present in the final population.

As used herein, “substantially pure” refers to free of contaminating molecules of unlike nature. “Substantially pure” also refers to a population of cells which it is at least 50% homogenous.

In a preferred embodiment, said population of cells is derived from CNS (central nervous system) tissue.

As used herein, “derived from” refers to “originating from”

As used herein, “CNS” refers to the part of the nervous system which, in vertebrates, consists of the brain and spinal cord, to which sensory impulses are transmitted and from which the motor impulses pass out, and which supervises and coordinates the activity of the entire nervous system.

In another preferred embodiment, the population of cells is derived from a cell culture. Methods of culturing cells are well-known in the art. Conditions for culturing a cell useful according to the invention are also known in the art and will vary depending on the cell being used. A cell that is cultured, according to the invention, is propagated or nurtured by incubation for a period of time, in an environment, and under conditions which support cell viability or propagation. A cell that is cultured may be subjected to one or more of the steps of expanding and proliferating the cell.

In another preferred embodiment, Sox gene expression is detected by nucleic acid hybridization.

As used herein, “expression” refers to production of a polypeptide or a nucleic acid (for example a Sox polypeptide or nucleic acid). The expression of a polypeptide can be detected according to methods well known in the art, for example immunoprecipitation, Western blot analysis or FACS analysis. The expression of a nucleic acid can be detected according to methods well known in the art, for example gel electrophoresis or by hybridization. Preferably, expression refers to an amount of production of a molecule (i.e., a protein or nucleic acid) that is detectable or measurable.

As used herein, “detecting” refers to determining the presence of a particular polypeptide, for example in a cell or on a cell surface. “Detecting” also refers to determining the presence of a nucleic acid in a cell or a sample. The amount of a polypeptide or nucleic acid that can be detected is preferably about 1 molecule to 10²⁰ molecules, more preferably about 100 to 1017 molecules and most preferably about 1000 to 10¹⁴ molecules. Methods well known in the art and described herein, can be used to detect or measure the presence or amount of a labeled or unlabeled polypeptide. Such methods include immunoprecipitation, Western blot analysis, FACS analysis, ELISA etc. . . . Methods well known in the art and described herein, can be used to detect or measure the presence or amount of a labeled or unlabeled nucleic acid. Such methods include gel electrophoresis followed by ethidium bromide staining, Northern or Southern blot hybridization analysis or in situ analysis. In embodiments wherein a polypeptide or nucleic acid to be detected is labeled, the method for detecting or measuring the polypeptide will be appropriate for measuring or detecting the label present on the polypeptide. The detection methods described herein are operative when as little as 1 or 2 molecules (and up to 1 or 2 million, for example 10, 100, 1000, 10,000, 1 million molecules) of polypeptide or nucleic acid are to be detected.

As used herein, “nucleic acid hybridization” refers to hydrogen bonding between two complementary nucleic acids sequences. As used herein, “stably hybridized” refers to a pair of nucleic acid sequences that associate with each other with a dissociation constant (K_{D}) of at least about 1×10³ M⁻¹, usually at least 1×10⁴ M⁻¹, typically at least 1×10⁵ M⁻¹, and preferably at least 1×10⁶ M⁻¹ to 1×10⁷ M⁻¹ or more.

As used herein, complementary refers to base pairs that bind to each other by hydrogen bonds. Adenine (A) and thymine (T) are complementary base pairs. Cytosine (C) and guanine (G) are also complementary base pairs. As used herein, “complementary” also refers to nucleic acid sequences that can bind to each other by hydrogen bonds between complementary base pairs. For example, the sequences 5′-TCGCAT-3′ and 3′-AGCGTA-5′ are completely complementary according to the invention. The invention also provides for sequences that are partially complementary.

As used herein, “partially complementary” refers to sequences that are less than 100% (i.e., 99%, 90%, 80%, 70%, 60%, 50% etc. . . . ) complementary.

In another preferred embodiment, Sox gene expression is detected by binding of a SOX polypeptide or a Sox nucleic acid corresponding to mRNA to a detectable ligand.

As used herein, a “nucleic acid corresponding to mRNA” refers to a nucleic acid molecule comprising the sequence of an mRNA molecule, for example a synthetic oligonucleotide or cDNA.

As used herein, “binding” or “association” refers to a polypeptide and a detectable ligand having a binding constant sufficiently strong to allow detection or binding by a detection means that is appropriate for the detectable ligand (for example FRET, autoradiography, western blot analysis, FACS, gel shift analysis etc . . . ), wherein the polypeptide and detectable ligand are in physical contact with each other and have a dissociation constant (Kd) of about 10 μM or lower.

A detectable ligand includes but is not limited to an antibody or antigen that is labeled, a labeled protein or nucleic acid that binds specifically to the polypeptide etc. . . .

In another preferred embodiment, the detectable ligand is a labeled immunoglobulin.

In another preferred embodiment, the detectable ligand is a labeled oligonucleotide complementary to Sox mRNA.

In another preferred embodiment, Sox gene expression is detected by FACS analysis.

According to a second aspect of the invention, cells can be actively sorted from other cell types by detecting the expression of SOX polypeptides in vivo using a reporter system. Thus, for example, the invention provides a method for isolating a desired cell type from a population of cells, comprising the steps of:

(a) transfecting the population of cells with a genetic construct comprising a coding sequence encoding a detectable marker operatively linked to Sox control regions;

(b) detecting the cells which express the selectable marker; and

(c) sorting the cells which express the selectable marker from the population of cells.

The selectable marker may be any selectable entity, including one which can be selected for with drugs such as antibiotics, but is preferably a fluorescent or luminescent marker which may be detected and sorted by automated cell sorting approaches. For example, the marker may be GPF or luciferase. Other useful markers include those which are expressed in the cell membrane, thus facilitating cell sorting by affinity means. Useful selectable markers also include beta-galactosidase, luciferase, and chloramphenical transferase.

Sox control sequences are control sequences derived from Sox genes and which regulate the expression of SOX polypeptides. By “regulate” is meant increase or decrease the expression or a SOX polypeptide. Preferably, a Sox control sequence increases expression of a SOX polypeptide by at least 2-fold, preferably 2-5 fold, more preferably 5-25 fold and most preferably 25-fold or more (for example 50-fold, 100-fold, 1000-fold, 10,000-fold or more) as compared to the level of expression of a SOX polypeptide from a nucleic acid encoding a SOX polypeptide that lacks Sox control sequences. In certain embodiments, a Sox control sequence increases or decreases expression of a SOX polypeptide by at least 5%, preferably 5-25%, more preferably 25-50% and most preferably 50-100%, as compared to the level of expression of a SOX polypeptide from a nucleic acid encoding a SOX polypeptide that lacks Sox control sequences. In certain embodiments, the activity of a Sox control sequence is dependent upon the presence of at least one regulatory factor that can alter (either increase or decrease) the activity of the Sox control sequence. “Regulate” also refers to control the timing of expression. For example, a marker gene that is operatively linked to a Sox control sequence may only be expressed in a cell simultaneously with a Sox gene, or at the same time during cell culture or cellular differentiation or development that a Sox gene would normally be expressed. Sox control sequences are nucleic acid sequences that are known in the art, as further described below. As used herein, “control sequences” or “control regions” refer to DNA sequences which are located either 5′ of the transcription start site, 3′ of the transcription termination site, within an intron or exon, and are capable of ensuring that the gene is transcribed at the proper time and in the appropriate cell type. Control sequences include promoter and enhancer sequences and sequences recognized by transcription factors and other DNA binding proteins.

According to a further aspect of the invention, cells can be actively sorted from other cell types by detecting the expression of SOX polypeptides in vivo using a reporter system which is itself responsive to Sox gene expression. Thus, for example, the invention provides a method for isolating a desired cell type from a population of cells, comprising the steps of:

(a) transfecting the population of cells with a genetic construct comprising a coding sequence encoding a detectable marker operatively linked to control regions sensitive, to modulation by a SOX polypeptide;

(b) detecting the cells which express the selectable marker; and

(c) sorting the cells which express the selectable marker from the population of cells.

As used herein, a “desired cell type” refers to any cell type of any lineage or capable of differentiating to any lineage. In certain embodiments, a “desired cell type” of the invention expresses a SOX polypeptide.

The expression of a gene of interest that is operatively linked to a “control region sensitive to modulation by a SOX polypeptide”, is “regulated” (either increased, decreased or expressed in a temporally distinct pattern from the pattern observed in the absence of binding of the SOX polypeptide to the SOX binding site) by a SOX polypeptide. When operatively linked to a gene of interest, “control regions sensitive to modulation by a SOX polypeptide” increase or decrease the level of expression or the temporal regulation of expression of the gene of interest in the presence of a SOX polypeptide. For example, the level of expression of a detectable marker operatively linked to a control region sensitive to modulation by a SOX polypeptide may be increased by at least 2-fold, 5, 10, 100, 1000, 10,000-fold or more in the presence of a SOX polypeptide, as compared to the level of expression in the absence of a SOX polypeptide. In another embodiment, the level of expression of a detectable marker operatively linked to a control region sensitive to modulation by a SOX polypeptide may be increased or decreased by 5, 10-20, 25-50, or 50-100% in the presence of a SOX polypeptide, as compared to the level of expression in the absence of a SOX polypeptide. In certain embodiments, “control regions sensitive to modulation by a SOX polypeptide” at a minimum comprise a SOX binding site, for example having the sequence A/T A/T CAA A/T G of the Sox1 binding site, or of any SOX binding site known in the art. A “SOX binding site” refers to a nucleic acid sequence to which a SOX polypeptide can bind, as defined herein. Preferably, as a result of the binding of a SOX polypeptide to a SOX binding site, the expression of a gene of interest that is operatively linked to a “control region sensitive to modulation by a SOX polypeptide”, is “regulated” (either increased, decreased or expressed in a temporally distinct pattern from the pattern observed in the absence of binding of the SOX polypeptide to the SOX binding site).

A genetic construct according to the invention may comprise any promoter and enhancer elements as required, so long as the overall control remains sensitive to a SOX polypeptide; in other words, no expression of the marker coding. sequence should take place in the absence of the desired SOX protein. The regulatory sequences responsive to SOX polypeptides are known in the art and have been described in the literature cited herein and incorporated herein by reference; at a minimum, however, the construct of the invention will comprise a SOX binding site. Preferably, the natural SOX-responsive control elements are used in their entirety; however, other promoter and enhancer elements may be substituted where they remain under the influence of SOX expression.

The selectable marker will only be expressed in desired cell types because only these cells express the relevant SOX polypeptide, which is required for transcription from the Sox control sequences. Preferably, therefore, the expression means used to express the selectable marker is not leaky and only a minimal amount of the marker (i.e., less than 5% of the amount of marker expressed in the presence of the SOX polypeptide) is expressed in the absence of the SOX polypeptide. Techniques for transforming cells with coding genetic constructs according to the invention, detecting the marker and sorting cells accordingly are known in the art.

The invention also provides for a method of isolating a neuroblastic cell from a population of cells comprising the steps of;

(a) transfecting the population of cells with a genetic construct comprising a coding sequence encoding a detectable marker operatively linked to a control sequence which is transactivatable by a SOX polypeptide;

(b) detecting the cells which express said selectable marker; and

(c) sorting the cells which express the selectable marker from the population of cells.

The expression of a gene of interest that is operatively linked to a “control sequence which is transactivatable by a SOX polypeptide”, is “regulated” (either increased, decreased or expressed in a temporally distinct pattern from the pattern observed in the absence of binding of the SOX polypeptide to the SOX binding site) in the presence of a SOX polypeptide. When operatively linked to a gene of interest, a “control sequence which is transactivatable by a SOX polypeptide” increases or decreases the level of expression or the temporal regulation of expression of the gene of interest in the presence of a SOX polypeptide. For example, the level of expression of a detectable marker operatively linked to a control sequence which is transactivatable by a SOX polypeptide may be increased by at least 2-fold, 5, 10, 100, 1000, 10,000-fold or more in the presence of a SOX polypeptide, as compared to the level of expression in the absence of a SOX polypeptide. In another embodiment, the level of expression of a detectable marker operatively linked to a control region sensitive to modulation by a SOX polypeptide may be increased or decreased by 5, 10-20, 25-50, or 50-100% in the presence of a SOX polypeptide, as compared to the level of expression in the absence of a SOX polypeptide. In certain embodiments, a “control sequence which is transactivatable by a SOX polypeptide” at a minimum comprises a SOX binding site, for example having the sequence A/T A/T CAA A/T G of the Sox1 binding site, or of any SOX binding site known in the art. A “SOX binding site” refers to a nucleic acid sequence to which a SOX polypeptide can bind, as defined herein. Preferably, as a result of the binding of a SOX polypeptide to a SOX binding site, the expression of a gene of interest that is operatively linked to a “control sequence that is transactivatable by a SOX polypeptide”, is “regulated” (either increased, decreased or expressed in a temporally distinct pattern from the pattern observed in the absence of binding of the SOX polypeptide to the SOX binding site).

As used herein, a “neuroblastic cell” refers to a cell that is commited to develop into a neuron or neural cell. Preferably, a “neuroblastic cell” will differentiate into a neuronal cell that expresses at least one of the neuronal markers neurofilament light and heavy chains, synapsin, microtubule-associated proteins MAP2 and tau, or beta-tubulin III, NCAM, intermediate filament NESTIN, MASH1 and WNT1.

In a preferred embodiment, the selectable marker is a fluorescent or luminescent polypeptide.

The present invention, in a still further aspect, provides the use of Sox coding sequences to transform precursor cells and thereby differentiate desired partially committed cells therefrom. Accordingly, there is provided a method for differentiating partially committed cell from a pluripotent precursor cell, comprising the steps of:

(a) transforming the pluripotent precursor cell with a genetic construct comprising a Sox coding sequence operatively linked to a suitable control sequences; and

(b) culturing the cells so as to allow expression of the Sox coding sequence, thereby inducing the cell to differentiate.

As used herein, “differentiation” refers to the process by which a cell undergoes a change to a particular cell type, e.g. to a specialized cell type, for example a neural cell. Differentiation is usually accomplished by altering the expression of one or more genes of the progenitor cell and results in the cell altering its structure and function.

As used herein, a “Sox coding sequence” refers to a nucleic acid sequence that in its native state or in a recombinant form can be transcribed and/or translated to produce a SOX mRNA and/or the SOX polypeptide or a fragment thereof. As used herein, “coding region” or “coding sequence” refers to a region of DNA which encodes a protein, also known as an exon. A “Sox coding sequence” includes any of the coding sequences corresponding to the Sox gene sequences provided herein in the section entitled “Detailed Description of the Invention”.

As used herein, “non-coding region” refers to a region of DNA which does not encode a protein coding region, also known as an intron, and is not included in the RNA molecule that is synthesized from a particular gene.

As used herein, “culturing” refers to propagating or nurturing a cell, collection of cells, tissue, or organ, by incubating for a period of time in an environment and under conditions which support cell viability or propagation. Culturing can include one or more of the steps of expanding and proliferating a cell, collection of cells, tissue, or organ according to the invention.

In a preferred embodiment, the Sox coding sequence expressing a SOX polypeptide is operatively linked to an inducible promoter.

As used herein, an “inducible promoter” refers to a promoter that is only expressed in the presence of an exogenous or endogenous chemical (for example an alcohol, a hormone, or a growth factor), or in response to developmental changes or at particular stages of differentiation.

In another preferred embodiment, the cell is further transfected with a vector comprising a sequence encoding a regulator which regulates the expression of the Sox sequence.

As used herein, a “regulator” includes a protein, a nucleic acid, or any chemical compound that “regulates”, as defined herein, the expression of a Sox sequence. In certain embodiments, the regulator can bind directly to the Sox sequence or to a Sox regulatory sequence. In other embodiments, a regulator of the invention does not bind directly to the Sox sequence or to a Sox regulatory sequence.

In another preferred embodiment, the Sox gene is a member of Sox Group A.

In another preferred embodiment, the Sox gene is Sox1 or Sox2.

Suitable control sequences for use in the latter aspect of the invention are known in the art and may include inducible or constitutive control sequences. Inducible control sequences have the advantage that Sox gene expression may be switched off when desired, for example once the cell is to be differentiated into a more mature state.

Precursor cells may be, for example, ES cells, such as human ES cells and cells with similar pluripotent properties derived from germ cells (EG cells). More specific pluripotent precursors or direct precursors of any desired cell lineage may also be employed.

### DETAILED DESCRIPTION OF THE INVENTION

The present invention is directed to methods for isolating, or producing, cells of any desired lineage. The expression of Sox genes is associated with a wide variety of cell types. Table 1 is a non-exhaustive list of known Sox genes, and shows the cell lineages with which they are associated in vivo.

The temporal and tissue-specific expression patterns of Sox genes are the subject of study by many groups, and in many cases such patterns are well mapped. For example, Sox1 expression appears to be limited to the neural plate and in induction of lens-associated gene expression in the eye. Sox2 is more widespread in its expression patterns, being expressed widely in the preimplantation embryo, and effectively defining the totipotent lineage. During gastrulation it is turned off in the mesoderm, but remains active in prospective neuroectoderm.

TABLE 1
The SOX Gene Family

| | SPECIES | CHROMOSOME | MUTATIONS | EXPRESSION | COMMENTS |
|---|---|---|---|---|---|
| Sry | human | mammalian Y | sex reversal | genital ridge | testis determining factor |
| | mouse | | | others | |
| | marsupial | | | | |
| | others | | | | |
| Sox1 | human | human 12q34 | mouse KO | CNS, UGR | regulates crystallin genes |
| | mouse | mouse 8A | lens defects | lens | role in natural determination |
| | | | seizures | | |
| Sox2 | human | human eq27 | | CNS, UGR | regulates expression of FGF4 |
| | sheep | sheep 1q33 | | lens, PNS | and crystallin, interacts |
| | mouse | goat 1q33 | | gut, others | with OCT3/4 |
| | chick | | | | |
| | Xenopus | | | | |
| | others | | | | |
| Sox3 | human | muan Xq24 | Borjeson- | CNS, UGR, | |
| | marsupial | mouse X | Foresman- | oocytes | |
| | | | Lehmann | | |
| | | | syndrome? | | |
| Sox4 | human | human 6p21 | mouse KO | lymphocytes | |
| | mouse | | heart and | heart, others | |
| | | | B cell defects | | |
| Sox5 | human | human 12p12 | | spermatid | bends DNA, multiple forms |
| | mouse | | | brain | |
| Sox6 | mouse | mouse 7 | | CNS, testis | trout Sox6 is called SOX-LZ |
| | trout | | | | |
| Sox7 | Xenopus | | | various | |
| Sox9 | human | human 17q24 | Campomelic | pre-cartilage | mutations cause sex reversal, |
| | pig | mouse 11 | Dysplasia | CNS, | momental retardation and |
| | mouse | | | UGR, | bone malformation |
| | chick | | | testis | |
| | trout | | | | |
| Sox10 | human | human 22q13 | Dom mouse | neural crest | mutations cause |
| | mouse | mouse 15 | Waardenburg- | Schwann | multiple neural crest |
| | rat | | Hirschsprung | cells | developmental defects |
| | | | disease in | | |
| | | | humans | | |
| Sox11 | human | human 2p25 | | CNS, PNS | |
| | mouse | | | kidney, lung, | |
| | rat | | | oocytes, glla, | |
| | | | | others | |
| Sox12 | Xenopus | | | ovaries, others | |
| Sox13 | mouse | | | arteries, ovaries, | |
| | | | | kidneys, others | |
| Sox14 | human | human 3q22 | | CNS | |
| | mouse | mouse 9 | | | |
| | chick | | | | |
| Sox17 | mouse | mouse 1 | dominant | testis, lung | Xsox17 responds to activin |
| | Xenopus | | negative in | endoderm | and induces endoderm markers |
| | | | Xenopus | | |
| Sox18 | human | mouse 2 | | lung, heart | human SOX18 binds 1 g enhancer |
| | mouse | | | muscle, | |
| | | | | B-cells | |
| Sox19 | zebrafish | | | CNS, lens, | |
| | | | | retina, B-cells | |
| Sox20 | human | human 17p13 | | fibroblasts, | |
| | | | | lymphoblasts, | |
| | | | | testis | |
| Sox21 | chick | | | CNS | |
| Sox22 | human | human 20p13 | | CNS, others | |
| Sox23 | trout | | | ovary, brain | binds nucleoprotein p62 protein |
| Sox24 | trout | | | oocytes | |
| XLS13A | Xenopus | | | oocytes | two closely related proteins, |
| XLS13B | | | | testes | very similar to Xsox11 |
| | | | | others | |
| SoxD | Xenopus | | dominant- | ectoderm, | role in neural induction |
| | | | negative | CNS | |
| | | | made | | |
| Sox70D/ | Drosophila | | fish-hook | zygote, CNS | role in CNS midline embryo |
| dichaete/ | | | and dichaete | | segmentation |
| fish-hook | | | mutants | | |

At this stage of differentiation, therefore, Sox2 has become a marker for cells committed to the neural lineage, but still capable of differentiation into a variety of cell types within that lineage. However, it is also expressed in gut endoderm, in cells lining the developing lung and ectodermal lineages which give rise to eye, olfactory and ear tissues, and in hair follicle tissues of mesodermal and ectodermal origins.

Sox3 is expressed throughout the ectoderm before gastrulation, and then becomes largely restricted to the neuroectoderm, as with Sox1 and Sox2. Although not as widely expressed as Sox2, it does retain expression at some mesodermal locations.

Sox4 is expressed in embryonic heart and spinal chord, and adult pre-B and T lymphocytes.

The method of the invention does not require absolutely unique expression of a Sox gene in order to isolate partially committed pluripotent cells. The present invention provides that Sox genes in general are markers for the state of pluripotency, rather than for any particular tissue. Accordingly, tissues or cell types may be sorted, for example by dissection of relevant tissues from embryos, or by induction of differentiation in cells in order to produce suitable cell populations; Sox gene expression may then be used to detect a pluripotent cell type in the selected population of cells. For example, Sox2 is associated with ES cells, Sox1, 2 and 3 with neural stem, cells, Sox9 with chondrocytes and Sox2 with hair follicle cells.

At least the following Sox genes are known; others may be isolated by homology searching. Sox21 (GenBank Accession No. AF107044); Sox14 (GenBank Accession No. 107043); Sox13 (GenBank Accession No. AB104474); Sox10 (GenBank Accession No. AJ001183); Sox22 (GenBank Accession No. U35612); Sox18 (GenBank Accession No. L35032); Sox11 (GenBank Accession No. U23752); Sox1 (GenBank Accession No. Y13436); Sox2 (GenBank Accession No. Z31560 and U12532); Sox3 (GenBank Accession No. X94125); Sox4 (GenBank Accession No. X70683); Sox5 (GenBank Accession No. S83306); Sox6 (GenBank Accession No. U32614); Sox7 (GenBank Accession No. AI15903/P40646); Sox9 (GenBank Accession No. S74504/5/6); Sox12 (GenBank Accession No. U70442); Sox13 (GenBank Accession No. AB006329); Sox15 (GenBank Accession No. AB104474); Sox16 (GenBank Accession No. L29084); Sox17 (GenBank Accession No. D49473); Sox19 (GenBank Accession No. X98368); Sox22 (GenBank Accession No. U35612).

Sox genes are divisible into subfamilies, based on homologies in the HMG box. Sox1, 2 and 3 belong to a single subfamily, Group B. Expression of these three genes has been evolutionarily conserved. The Drosophila (Nambu & Nambu 1996; Russel et al., 1996) zebrafish (Vriz et al., 1996) Xenopus (Misuseki, 1998) and avian (Unwanogho et al., 1995; Streit et al., 1997; Rex et al., 1997) putative orthologues of Sox1, Sox2 and Sox3 all show expression throughout the neural primordium. Thus, Sox1, Sox2 and Sox3 represent a novel subgroup of transcription factors which can serve as general early neuroepithelial markers. The grouping of Sox genes is described in Bowles et al., Dev. Biol. 2000, 227:239-555.

In general, Sox proteins and genes as referred to herein may be derived from any source, preferably from a mammalian source such as human or mouse, but also from other sources. such as fish, bird, reptile, amphibian, sea urchin, roundworm (e.g. ceanohabditis elegans) and insect.

A number of Sox gene sequences are known in the art and provided under the GenBank accession numbers given above. Other Sox sequences may be isolated, for example from genomic or cDNA libraries, by conventional techniques. The sequences provided herein may be used as probes, or to prepare antibodies or other molecules capable of recognizing specific polypeptides. Preferably, the sequences used as probes are substantially homologous to the sequences provided herein.

“Substantial homology”, where homology indicates sequence identity, means more than 40% sequence-identity, preferably more than 45% sequence identity and most preferably a sequence identity of 50% or more. Advantageously, the sequence identity may be up to about 90 or 95%.

Sequence homology (or identity) may be determined using any suitable homology algorithm, using for example default parameters. Advantageously, the BLAST algorithm is employed, with parameters set to default values. The BLAST algorithm is described in detail at http://www.ncbi.nih.gov/BLAST/blast_help.html, which is incorporated herein by reference. The search parameters are defined as follows, and are advantageously set to the defined default parameters.

BLAST (Basic Local Alignment Search Tool) is the heuristic search algorithm employed by the programs blastp, blastn, blastx, tblastn, and tblastx; these programs ascribe significance to their findings using the statistical methods of Karlin & Altschul (1990, 1993) with a few enhancements. The BLAST programs were tailored for sequence similarity searching, for example to identify homologues to a query sequence. The programs are not generally useful for motif-style searching. For a discussion of basic issues in similarity searching of sequence databases, see Altschul et al. (1994).

The five BLAST programs available at http://www.ncbi.nlm.nih.gov/BLAST perform the following tasks:

blastp compares an amino acid query sequence against a protein sequence database;

blastn compares a nucleotide query sequence against a nucleotide sequence database;

blastx compares the six-frame conceptual translation products of a nucleotide query sequence (both strands) against a protein sequence database;

tblastn compares a protein query sequence against a nucleotide sequence database dynamically translated in all six reading frames (both strands);

tblastx compares the six-frame translations of a nucleotide query sequence against the six-frame translations of a nucleotide sequence database.

BLAST uses the following search parameters:

HISTOGRAM Display a histogram of scores for each search; default is yes. (See parameter H in the BLAST Manual.)

DESCRIPTIONS Restricts the number of short descriptions of matching sequences reported to the number specified; default limit is 100 descriptions. (See parameter V in the manual page.) See also EXPECT and CUTOFF.

ALIGNMENTS Restricts database sequences to the number specified for which high-scoring segment pairs (HSPs) are reported; the default limit is 50. If more database sequences than this satisfy the statistical significance threshold for reporting (see EXPECT and CUTOFF below), only the matches ascribed the greatest statistical significance are reported. (See parameter B in the BLAST Manual.)

EXPECT The statistical significance threshold for reporting matches against database sequences; the default value is 10, such that 10 matches are expected to be found merely by chance, according to the stochastic model of Karlin & Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual.)

CUTOFF Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual.) Typically, significance thresholds can be more intuitively managed using EXPECT.

MATRIX Specify an alternate scoring matrix for BLASTP, BLASTX, TBLASTN and TBLASTX. The default matrix is BLOSUM62 (Henikoff & Henikoff, 1992). The valid alternative choices include: PAM40, PAM 120, PAM250 and IDENTITY. No alternate scoring matrices are available for BLASTN; specifying the MATRIX directive in BLASTN requests returns an error response.

STRAND Restrict a TBLASTN search to just the top or bottom strand of the database sequences; or restrict a BLASTN, BLASTX or TBLASTX search to just reading frames on the top or bottom strand of the query sequence.

FILTER Mask off segments of the query sequence that have low compositional complexity, as determined by the SEG program of Wootton & Federhen (Computers and Chemistry, 1993), or segments consisting of short-periodicity internal repeats, as determined by the XNU program of Claverie & States (Computers and Chemistry, 1993), or, for BLASTN, by the DUST program of Tatusov & Lipman (in preparation). Filtering can eliminate statistically significant but biologically uninteresting reports from the blast output (e.g. hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g. “NNNNNNNNNNNNN”) and the letter “X” in protein sequences (e.g. “XXXXXXXXX”). Users may turn off filtering by using the “Filter” option on the “Advanced options for the BLAST server” page.

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCB1-gi causes NCB1-gi identifiers to be shown in the output, in addition to the accession and/or locus name.

Most preferably, sequence comparisons are conducted using the simple BLAST search algorithm provided at http://www.ncbi.nlm.nih.gov/BLAST.

Preferably, the invention makes use of fragments of Sox sequences. As used herein, a fragment refers to a portion of a sequence comprising less than then entire genomic or cDNA sequence, for example 99%, 90%, 80%, 50%, 10% etc . . . of the sequence of a Sox gene or cDNA sequence. Fragments of the nucleic acid sequence of a few nucleotides in length, preferably 5 to 150 nucleotides in length, are especially useful as probes.

Exemplary nucleic acids, including those of new Sox clones derived according to the invention can alternatively be characterized as those nucleotide sequences which encode a SOX protein and hybridize to the DNA sequences set forth above, or a selected fragment of said DNA sequences. Preferred are such sequences encoding SOX polypeptides which hybridize under high-stringency conditions to the sequence set forth above.

Stringency of hybridization refers to conditions under which polynucleic acids hybrids are stable. Such conditions are evident to those of ordinary skill in the field. As known to those of skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridization reaction is performed under conditions of higher stringency, followed by washes of varying stringency.

As used herein, high stringency refers to conditions that permit hybridization of only those nucleic acid sequences that form stable hybrids in 1 M Na+ at 65-68° C. High stringency conditions can be provided, for example, by hybridization in an aqueous solution containing 6× SSC, 5× Denhardt's, 1% SDS (sodium dodecyl sulphate), 0.1 Na+pyrophosphate and 0.1 mg/ml denatured salmon sperm DNA as non specific competitor. Following hybridization, high stringency washing may be done in several steps, with a final wash (about 30 min) at the hybridization temperature in 0.2-0.1× SSC, 0.1% SDS.

Moderate stringency refers to conditions equivalent to hybridization in the above described solution but at about 60-62° C. In that case the final wash is performed at the hybridization temperature in 1× SSC, 0.1% SDS.

Low stringency refers to conditions equivalent to hybridization in the above described solution at about 50-52° C. In that case, the final wash is performed at the hybridization temperature in 2× SSC, 0.1% SDS.

It is understood that these conditions may be adapted and duplicated using a variety of buffers, e.g. formamide-based buffers, and temperatures. Denhardt's solution and SSC are well known to those of skill in the art as are other suitable hybridization buffers (see e.g. Sambrook et al., eds. (1989) Molecular Cloning: A Laboratory Manual, Cold Spring Harbor Laboratory Press, New York or Ausubel et al., eds. (1990) Current Protocols in Molecular Biology, John Wiley & Sons, Inc.). Optimal hybridization conditions have to be determined empirically, as the length and the GC content of the hybridizing pair also play a role.

Typically, selective hybridization occurs when two nucleic acid sequences are substantially complementary (at least about 65% complementary over a stretch of at least 14 to 25 nucleotides, preferably at least about 75%, more preferably at least about 90% complementary). See Kanehisa, M., 1984, Nucleic Acids Res. 12: 203, incorporated herein by reference. As a result, it is expected that a certain degree of mismatch at the priming site is tolerated. Such mismatch may be small, such as a mono-, di- or tri-nucleotide. Alternatively, a region of mismatch may encompass loops, which are defined as regions in which there exists a mismatch in an uninterrupted series of four or more nucleotides.

Numerous factors influence the efficiency and selectivity of hybridization of a first nucleic acid to a second nucleic acid molecule. These factors, which include nucleic acid length, nucleotide sequence and/or composition, hybridization temperature, buffer composition and potential for steric hindrance in the region to which the primer is required to hybridize, will be considered when designing oligonucleotides according to the invention.

A positive correlation exists between nucleic acid length and both the efficiency and accuracy with which a first nucleic acid will anneal to a second nucleic acid. In particular, longer sequences have a higher melting temperature (T_{M}) than do shorter ones, and are less likely to be repeated within a given target sequence, thereby minimizing promiscuous hybridization. Nucleic acid sequences with a high G-C content or that comprise palindromic sequences tend to self-hybridize, as do their intended target sites, since unimolecular, rather than bimolecular, hybridization kinetics are generally favored in solution. However, it is also important to design a nucleic acid that contains sufficient numbers of G-C nucleotide pairings since each G-C pair is bound by three hydrogen bonds, rather than the two that are found when A and T bases pair to bind the target sequence, and therefore forms a tighter, stronger bond. Hybridization temperature varies inversely with nucleic acid annealing efficiency, as does the concentration of organic solvents, e.g. formamide, that might be included in a hybridization mixture, while increases in salt concentration facilitate binding. Under stringent annealing conditions, longer hybridization probes, or synthesis primers, hybridize more efficiently than do shorter ones, which are sufficient under more permissive conditions. Preferably, stringent hybridization is performed in a suitable buffer (for example, 1× Sentinel Molecular Beacon PCR Core buffer, Stratagene Catalog #600500; 1× Pfu buffer, Stratagene Catalog #200536; or 1× Cloned Pfu buffer, Stratagene Catalog #200532) under conditions that allow the first nucleic acid sequence to hybridize to the second nucleic acid sequence (e.g., 95° C.). Stringent hybridization conditions can vary (for example, salt concentrations may range from less than about 1M, more usually less than about 500 mM and preferably less than about 200 mM) and hybridization temperatures can range (for example, from as low as 0° C. to greater than 22° C., greater than about 30° C., and (most often) in excess of about 37° C.), depending upon the length and/or nucleic acid composition of the nucleic acids. Longer fragments may require higher hybridization temperatures for specific hybridization. As several factors affect the stringency of hybridization, the combination of parameters is more important than the absolute measure of a single factor.

Advantageously, the invention moreover provides nucleic acid sequences which are capable of hybridizing, under stringent conditions, to a fragment of a Sox gene as set forth above. Preferably, the fragment is between 15 and 50 bases in length. Advantageously, it is about 25 bases in length.

As will be appreciated by those skilled in the art, the redundancy of the genetic code allows the design of a large number of sequences encoding SOX polypeptides. Any of these sequences may be useful for expressing SOX polypeptides as described below. An advantage of the use of a sequence encoding human SOX1 which is not the endogenous human Sox1 sequence is that the mRNA produced has a different sequence to that of the endogenous SOX mRNA, and may thus be distinguished therefrom. Antisense oligonucleotides may be designed which are capable of selectively inhibiting the expression of either endogenous or exogenous Sox genes.

As used herein, “endogenous” refers to expressed or present naturally in a cell.

As used herein, “exogenous” refers to not expressed or present naturally in a cell.

Given the guidance provided herein, nucleic acids encoding SOX polypeptides are obtainable according to methods well known in the art. For example, a nucleic acid encoding SOX polypeptides is obtainable by chemical synthesis, using polymerase chain reaction (PCR) or by screening a genomic library or a suitable cDNA library prepared from a source believed to express SOX polypeptides and to express it at a detectable level.

Chemical methods for synthesis of a nucleic acid of interest are known in the art and include triester, phosphite, phosphoramidite and H-phosphonate methods, PCR and other autoprimer methods as well as oligonucleotide synthesis on solid supports. These methods may be used if the entire nucleic acid sequence of the nucleic acid is known, or the sequence of the nucleic acid complementary to the coding strand is available. Alternatively, if the target amino acid sequence is known, one may infer potential nucleic acid sequences using known and preferred coding residues for each amino acid residue.

An alternative means to isolate genes encoding SOX polypeptides is to use PCR technology as described e.g. in section 14 of Sambrook et al., 1989. This method requires the use of oligonucleotide probes that will hybridize to Sox nucleic acid. Strategies for selection of oligonucleotides are described below.

Libraries are screened with probes or analytical tools designed to identify the gene of interest or the protein encoded by it. For cDNA expression libraries suitable means include monoclonal or polyclonal antibodies that recognize and specifically bind to SOX polypeptides; oligonucleotides of about 20 to 80 bases in length that encode known or suspected Sox cDNA from the same or different species; and/or complementary or homologous cDNAs or fragments thereof that encode the same or a hybridizing gene. Appropriate probes for screening genomic DNA libraries include, but are not limited to oligonucleotides, cDNAs or fragments thereof that encode the same or hybridizing DNA; and/or homologous genomic DNAs or fragments thereof.

A nucleic acid encoding SOX polypeptides may be isolated by screening suitable cDNA or genomic libraries under suitable hybridization conditions with a probe, i.e. a nucleic acid disclosed herein including oligonucleotides derivable from the sequences set forth above. Suitable libraries are commercially available or can be prepared e.g. from cell lines, tissue samples, and the like.

As used herein, a probe is e.g. a single-stranded DNA or RNA that has a sequence of nucleotides that includes between 10 and 50, preferably between 15 and 30 and most preferably at least about 20 contiguous bases that are the same as (or the complement of) an equivalent or greater number of contiguous bases of a Sox gene set forth above. The nucleic acid sequences selected as probes should be of sufficient length and sufficiently unambiguous so that false positive results are minimized. The nucleotide sequences are usually based on conserved or highly homologous nucleotide sequences or regions of SOX polypeptides. The nucleic acids used as probes may be degenerate at one or more positions. The use of degenerate oligonucleotides may be of particular importance where a library is screened from a species in which preferential codon usage in that species is not known.

As used herein, “degeneracy” in a nucleic acid sequence refers to the lack of effect of many changes in a nucleotide encoding a codon (for example the nucleotide in the third base of the codon) on the amino acid that is represented.

Preferred regions from which to construct probes include 5′ and/or 3′ coding sequences, sequences predicted to encode ligand binding sites, and the like. For example, either the full-length cDNA clone disclosed herein or fragments thereof can be used as probes. Preferably, nucleic acid probes of the invention are labeled with suitable label means for ready detection upon hybridization. For example, a suitable label means is a radiolabel. The preferred method of labeling a DNA fragment is by incorporating α³²P dATP with the Klenow fragment of DNA polymerase in a random priming reaction, as is well known in the art. Oligonucleotides are usually end-labeled with γ³²P-labeled ATP and polynucleotide kinase. However, other methods (e.g. non-radioactive) may also be used to label the fragment or oligonucleotide, including e.g. enzyme labeling, fluorescent labeling with suitable fluorophores and biotinylation.

After screening the library e.g. with a portion of DNA including substantially the entire Sox1-encoding sequence or a suitable oligonucleotide based on a portion of said DNA, positive clones are identified by detecting a hybridization signal; the identified clones are characterized by restriction enzyme mapping and/or DNA sequence analysis, and then examined, e.g. by comparison with the sequences set forth herein, to ascertain whether they include DNA encoding a complete Sox1 cDNA sequence (i.e., if they include translation initiation and termination codons) or a complete gene sequence. As used herein, “substantially” as it refers to the entire Sox1 encoding sequence means at least 30%, preferably 30-50%, more preferably 50-80% and most preferably 80-100% of the entire Sox1 coding sequence. If the selected clones are incomplete, they may be used to rescreen the same or a different library to obtain overlapping clones. If the library is genomic, then the overlapping clones may include exons and introns. If the library is a cDNA library, then the overlapping clones will include an open reading frame. In both instances, complete clones may be identified by comparison with the DNAs and deduced amino acid sequences provided herein.

It is envisaged that SOX-encoding sequences can be readily modified by nucleotide substitution, nucleotide deletion, nucleotide insertion or inversion of a nucleotide stretch, and any combination thereof. Such mutants can be used e.g. to produce a mutant SOX polypeptide that has an amino acid sequence differing from the sequences of SOX polypeptides as found in nature. Mutagenesis may be predetermined (site-specific) or random. A mutation which is not a silent mutation must not place sequences out of reading frames and preferably will not create complementary regions that could hybridize to produce secondary mRNA structure such as loops or hairpins.

Sorting of cells, based upon detection of expression of Sox genes, may be performed by any technique known in the art, as exemplified above. For example, cells may be sorted by flow cytometry or FACS. For a general reference, see Flow Cytometry and Cell Sorting: A Laboratory Manual (1992) A. Radbruch (Ed.), Springer Laboratory, New York.

Flow cytometry is a powerful method for studying and purifying cells. It has found wide application, particularly in immunology and cell biology: however, the capabilities of the FACS method can be applied in many other fields of biology. The acronym F.A.C.S. stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (˜100%).

FACS machines collect fluorescence signals in one to several channels corresponding to different laser excitation and fluorescence emission wavelengths. Fluorescent labeling allows the investigation of many aspects of cell structure and function. The most widely used application is immunofluorescence: the staining of cells with antibodies conjugated to fluorescent dyes such as fluorescein and phycoerythrin. This method is often used to label molecules on the cell surface, but antibodies can also be directed at targets within the cell. In direct immunofluorescence, an antibody to a particular molecule, the SOX polypeptide, is directly conjugated to a fluorescent dye. Cells can then be stained in one step. In indirect immunofluorescence, the primary antibody is not labeled, but a second fluorescently conjugated antibody is added which is specific for the first antibody: for example, if the anti-SOX antibody is a mouse IgG, then the second antibody could be a rat or rabbit antibody raised against mouse IgG.

FACS can be used to measure gene expression in cells transfected with recombinant DNA encoding SOX polypeptides. This can be achieved directly, by labeling of the protein product, or indirectly by using a reporter gene in the construct. Examples of reporter genes are β-galactosidase and Green Fluorescent Protein (GFP). β-galactosidase activity can be detected by FACS using fluorogenic substrates such as fluorescein digalactoside (FDG). FDG is introduced into cells by hypotonic shock, and is cleaved by the enzyme to generate a fluorescent product, which is trapped within the cell. One enzyme can therefore generate a large amount of fluorescent product. Cells expressing GFP constructs will fluoresce without the addition of a substrate. Mutants of GFP are available which have different excitation frequencies, but which emit fluorescence in the same channel. In a two-laser FACS machine, it is possible to distinguish cells which are excited by the different lasers and therefore assay two transfections at the same time.

Alternative means of cell sorting may also be employed. For example, the invention comprises the use of nucleic acid probes complementary to Sox mRNA. Such probes can be used to identify cells expressing SOX polypeptides individually, such that they may subsequently be sorted either manually, or using FACS sorting. Nucleic acid probes complementary to Sox mRNA may be prepared according to the teaching set forth above, using the general procedures as described by Sambrook et al. (1989).

In a preferred embodiment, the invention comprises the use of an antisense nucleic acid molecule, complementary to a Sox mRNA, conjugated to a fluorophore which may be used in FACS cell sorting. Methods of designing and using antisense nucleic acid molecules are well-known in the art.

Suitable imaging agents for use with FACS may be delivered to the cells by any suitable technique, including simple exposure thereto in cell culture, delivery of transiently expressing nucleic acids by viral or non-viral vector means, liposome-mediated transfer of nucleic acids or imaging agents, and the like.

The invention, in certain embodiments, includes antibodies specifically recognizing and binding to SOX polypeptides. For example, such antibodies may be generated against the SOX polypeptides having the amino acid sequences set forth above. Alternatively, SOX polypeptides or fragments thereof (which may also be synthesized by in vitro methods) are fused (by recombinant expression or an in vitro peptidyl bond) to an immunogenic polypeptide and this fusion polypeptide, in turn, is used to raise antibodies against a SOX epitope.

Anti-SOX antibodies may be recovered from the serum of immunized animals. Monoclonal antibodies may be prepared from cells from immunized animals in the conventional manner.

The antibodies of the invention are useful for identifying SOX1 in neural cells expressing Sox1, in accordance with the present invention.

Antibodies according to the invention may be whole antibodies of natural classes, such as IgE and IgM antibodies, but are preferably IgG antibodies. Moreover, the invention includes antibody fragments, such as Fab, F(ab′)2, Fv and ScFv. Small fragments, such as Fv and ScFv, possess advantageous properties for diagnostic and therapeutic applications due to their small size and consequent superior tissue distribution.

The antibodies may comprise a label. Especially preferred are labels which allow the imaging of the antibody in neural cells in vivo. Such labels may be radioactive labels or radioopaque labels, such as metal particles, which are readily visualizable within tissues. Moreover, they may be fluorescent labels or other labels which are visualizable in tissues and which may be used for cell sorting.

Recombinant DNA technology may be used to improve the antibodies of the invention. Thus, chimeric antibodies may be constructed in order to decrease the immunogenicity thereof in diagnostic or therapeutic applications. Moreover, immunogenicity may be minimized by humanizing the antibodies by CDR grafting [see European Patent Application 0 239 400 (Winter)] and, optionally, framework modification.

Antibodies according to the invention may be obtained from animal serum, or, in the case of monoclonal antibodies or fragments thereof, produced in cell culture. Recombinant DNA technology may be used to produce the antibodies according to established procedure, in bacterial or preferably mammalian cell culture. The selected cell culture system preferably secretes the antibody product.

Therefore, the present invention includes a process for the production of an antibody according to the invention comprising culturing a host, e.g. E. coli or a mammalian cell, which has been transformed with a hybrid vector comprising an expression cassette comprising a promoter operably linked to a first DNA sequence encoding a signal peptide linked in the proper reading frame to a second DNA sequence encoding the protein, and isolating the protein.

Multiplication of hybridoma cells or mammalian host cells in vitro is carried out in suitable culture media, which are the customary standard culture media, for example Dulbecco's Modified Eagle Medium (DMEM) or RPMI 1640 medium, optionally replenished by a mammalian serum, e.g. fetal calf serum, or trace elements and growth sustaining supplements, e.g. feeder cells such as normal mouse peritoneal exudate cells, spleen cells, bone marrow macrophages, 2-aminoethanol, insulin, transferrin, low density lipoprotein, oleic acid, or the like. Multiplication of host cells which are bacterial cells or yeast cells is likewise carried out in suitable culture media known in the art, for example for bacteria in medium LB, NZCYM, NZYM, NZM, Terrific Broth, SOB, SOC, 2× YT, or M9 Minimal Medium, and for yeast in medium YPD, YEPD, Minimal Medium, or Complete Minimal Dropout Medium.

In vitro production provides relatively pure antibody preparations and allows scale-up to give large amounts of the desired antibodies. Techniques for bacterial cell, yeast or mammalian cell cultivation are known in the art and include homogeneous suspension culture e.g. in an airlift reactor or in a continuous stirrer reactor, or immobilized or entrapped cell culture e.g. in hollow fibers, microcapsules, on agarose microbeads or ceramic cartridges.

Large quantities of the desired antibodies can also be obtained by multiplying mammalian cells in vivo. For this purpose, hybridoma cells producing the desired antibodies are injected into histocompatible mammals to cause growth of antibody-producing tumors. Optionally, the animals are primed with a hydrocarbon, especially mineral oils such as pristane (tetramethyl-pentadecane), prior to the injection. After one to three weeks, the antibodies are isolated from the body fluids of those mammals. For example, hybridoma cells obtained by fusion of suitable myeloma cells with antibody-producing spleen cells from Balb/c mice, or transfected cells derived from hybridoma cell line Sp2/0 that produce the desired antibodies are injected intraperitoneally into Balb/c mice optionally pre-treated with pristane, and, after one to two weeks, ascitic fluid is taken from the animals.

The cell culture supernatants are screened for the desired antibodies, preferentially by immunofluorescent staining of cells expressing SOX polypeptides, by immunoblotting, by an enzyme immurioassay e.g. a sandwich assay or a dot-assay, or a radioimmunoassay.

For isolation of the antibodies, the immunoglobulins in the culture supernatants or in the ascitic fluid may be concentrated e.g. by precipitation with ammonium sulphate, dialysis against hygroscopic material such as polyethylene glycol, filtration through selective membranes, or the like. If necessary and/or desired, the antibodies are purified by the customary chromatography methods, for example gel filtration, ion-exchange chromatography, chromatography over DEAE-cellulose and/or (immuno-)affinity chromatography e.g. affinity chromatography with SOX protein or with Protein-A.

The invention further concerns hybridoma cells secreting the monoclonal antibodies of the invention. The preferred hybridoma cells of the invention are genetically stable, secrete monoclonal antibodies of the invention of the desired specificity and can be activated from deep-frozen cultures by thawing and recloning.

The invention also concerns a process for the preparation of a hybridoma cell line secreting monoclonal antibodies directed against SOX polypeptides, characterized in that a suitable mammal, for example a Balb/c mouse, is immunized with purified SOX protein, an antigenic carrier containing purified SOX polypeptide or with cells bearing SOX polypeptides. Antibody-producing cells of the immunized mammal are fused with cells of a suitable myeloma cell line, the hybrid cells obtained in the fusion are cloned and cell clones secreting the desired antibodies are selected. For example spleen cells of Balb/c mice immunized with cells bearing SOX polypeptides are fused with cells of the myeloma cell line PAI or the myeloma cell line Sp2/0-Ag14, the obtained hybrid cells are screened for secretion of the desired antibodies, and positive hybridoma cells are cloned.

Preferred is a process for the preparation of a hybridoma cell line, characterized in that Balb/c mice are immunized by injecting subcutaneously and/or intraperitoneally between 10 and 10⁷ and 10⁸ cells of human tumor origin which express SOX polypeptides containing a suitable adjuvant several times, e.g. four to six times, over several months, e.g. between two and four months, and spleen cells from the immunized mice are taken two to four days after the last injection and fused with cells of the myeloma cell line PAT in the presence of a fusion promoter, preferably polyethylene glycol. Preferably the myeloma cells are fused with a three- to twentyfold excess of spleen cells from the immunized mice in a solution containing about 30% to about 50% polyethylene glycol of a molecular weight around 4000. After the fusion the cells are expanded in suitable culture media as described hereinbefore, supplemented with a selection medium, for example HAT medium, at regular intervals in order to prevent normal myeloma cells from overgrowing the desired hybridoma cells.

The invention also concerns recombinant DNAs comprising an insert coding for a heavy chain variable domain and/or for a light chain variable domain of an antibody directed to the extracellular domain of a SOX polypeptide as described hereinbefore. By definition such DNAs comprise coding single stranded DNAs, double stranded DNAs consisting of said coding DNAs and of complementary DNAs thereto, or these complementary (single stranded) DNAs themselves.

Furthermore, DNA encoding a heavy chain variable domain and/or a light chain variable domain of an antibody directed against a SOX polypeptide can be enzymatically or chemically synthesized to have the authentic DNA sequence coding for a heavy chain variable domain and/or for the light chain variable domain, or for a mutant thereof. A mutant of the authentic DNA is a DNA encoding a heavy chain variable domain and/or a light chain variable domain of the above-mentioned antibodies in which one or more amino acids are deleted or exchanged with one or more other amino acids. Preferably said modification(s) are outside the CDRs of the heavy chain variable domain and/or of the light chain variable domain of the antibody. Such a mutant DNA is also intended to be a silent mutant wherein one or more nucleotides are replaced by other nucleotides with the new codons coding for the same amino acid(s). Such a mutant sequence is also a degenerate sequence. Degenerate sequences are degenerate within the meaning of the genetic code in that an unlimited number of nucleotides are replaced by other nucleotides without resulting in a change in the amino acid sequence originally encoded. Such degenerate sequences may be useful due to their different restriction sites and/or frequency of particular codons which are preferred by the specific host, particularly E. coli, to obtain an optimal expression of the heavy chain murine variable domain and/or a light chain murine variable domain.

As used herein “mutation” refers to a variation in the nucleotide sequence of a gene or regulatory sequence as compared to the naturally occurring or normal nucleotide sequence. A mutation may result from the deletion, insertion or substitution of more than one nucleotide (e.g., 2, 3, 4, or more nucleotides) or a single nucleotide change such as a deletion, insertion or substitution. The term “mutation” also encompasses chromosomal rearrangements.

As used herein, “alteration” refers to a change in either a nucleotide or amino acid sequence, as compared to the naturally occurring sequence, resulting from a deletion, an insertion or addition, or a substitution.

As used herein, “deletion” refers to a change in either nucleotide or amino acid sequence wherein one or more nucleotides or amino acid residues, respectively, are absent.

As used herein, “insertion” or “addition” refers to a change in either nucleotide or amino acid sequence wherein one or more nucleotides or amino acid residues, respectively, have been added.

As used herein, “substitution” refers to a replacement of one or more nucleotides or amino acids by different nucleotides or amino acid residues, respectively.

The term mutant is intended to include a DNA mutant obtained by in vitro mutagenesis of the authentic DNA according to methods known in the art.

For the assembly of complete tetrameric immunoglobulin molecules and the expression of chimeric antibodies, the recombinant DNA inserts coding for heavy and light chain variable domains are fused with the corresponding DNAs coding for heavy and light chain constant domains, then transferred into appropriate host cells, for example after incorporation into hybrid vectors.

The invention therefore also concerns recombinant DNAs comprising an insert coding for a heavy chain murine variable domain of an antibody directed against SOX polypeptides fused to a human constant domain g, for example γ1, γ2, γ3 or γ4, preferably γ1 or γ4. Likewise the invention concerns recombinant DNAs comprising an insert coding for a light chain murine variable domain of an antibody directed to SOX polypeptides fused to a human constant domain κ or λ chain, preferably κ.

In another embodiment the invention pertains to recombinant nucleic acids wherein the heavy chain variable domain and the light chain variable domain are linked by way of a DNA insert coding for a spacer group, optionally comprising a signal sequence facilitating the processing of the antibody in the host cell and/or a DNA coding for a peptide facilitating the purification of the antibody and/or a DNA coding for a cleavage site and/or a DNA coding for a peptide spacer and/or a DNA coding for an effector molecule, such as a label.

According to a further aspect, and as referred to above, neuroblastic cells may be actively sorted from other cell types by detecting Sox1 expression in vivo using a reporter system. For example, such a reporter system may comprise a readily identifiable marker under the control of a SOX activated expression system. Fluorescent markers, which can be detected and sorted by FACS, are preferred. Especially preferred are GFP and luciferase.

Alternatively, an in vivo construct expressing a reporter may be placed under the control of the Sox control sequences themselves. These sequences are activated at the same time as Sox expression is activated, and therefore mark the transition into the neural pathway with the same accuracy as the Sox gene of interest. Advantageously, the Sox control sequences used are vertebrate Sox control sequences, preferably human Sox control sequences.

In general, reporter constructs useful for detecting neural cells by expression of a reporter gene may be constructed according to the general teaching of Sambrook et al. (1989). Typically, constructs according to the invention comprise a promoter regulated by Sox1, and a coding sequence encoding the desired reporter, for example GFP or luciferase. Vectors encoding GFP and luciferase are known in the art and available commercially.

It is known that SOX proteins bind to a defined sequence motif. For example, Sox1 binds to A/T A/T CAA A/T G with high affinity. Accordingly, constructs according to the invention advantageously comprise SOX binding elements, or a functional equivalent thereof, operably linked to a gene encoding a selectable marker. As used herein, a “functional equivalent of a SOX binding element” comprises a nucleic acid sequence to which a SOX polypeptide can bind, as defined herein. Preferably, the expression of a gene of interest that is operatively linked to a functional equivalent of a SOX binding element is “regulated”, as defined herein, when a SOX polypeptide is bound to the functional equivalent of a SOX binding element.

When a construct comprising a SOX binding element or a functional equivalent thereof is transfected into cells which potentially express SOX polypeptides, these constructs according to the invention will be activated specifically by SOX polypeptide expression. Therefore, the selectable marker will be expressed once the cell enters the desired differentiation state which correlates with expression of the relevant SOX polypeptide. This allows cells entering the neural differentiation pathway to be sorted by FACS.

In a still further aspect, the present invention relates to the transfection of pluripotent precursor cells, capable of differentiating into cells of a desired lineage, with a vector expressing a SOX polypeptide. By such means, pluripotent precursor cells may be induced to differentiate along a desired pathway, becoming partially committed cells capable of differentiating into a variety of specialized tissues.

Herein, terms such as “transfection”, “transformation” and the like are not intended to be significant, except to indicate that nucleic acid is transferred to a cell or organism in functional form. Such terms include various means of transferring nucleic acids to cells, including transfection with CaPO₄, electroporation, viral transduction, lipofection, delivery using liposomes and other delivery vehicles, biolistics and the like. Such techniques are well-known in the art.

Suitable pluripotent precursor cells may be derived from a number of sources. For example, ES cells, such as human ES cells and cells derived from Germ cells (EG cells) may be derived from embryonal tissue and cultured as cell lines (Thomson et al., (1998) Science 282:1145-1147). Alternatively, pluripotent cells may be prepared by retrodifferentiation, by the administration of growth factors or otherwise, or by cloning, such as by nuclear transfer from an adult cell to a pluripotent cell such as an ovum.

Human stem cells of specific lineages may be isolated from human tissues directly. Alternatively, stem sells from non-human animals, such as rodents, may be used.

Stem cells may also be propagated in vitro, for example as described in Snyder et al., (1996) Clinical Neuroscience 3:310-316, and Martinez-Serrano et al., (1996) Clinical Neuroscience 3:301-309. Moreover, pluripotent cell lines, such as the N-Tera II cell line which are capable of differentiating into neural cells upon stimulation with agents such as retinoic acid, also express Sox genes and are useful according to the invention.

The cDNA or genomic DNA encoding native or mutant SOX polypeptides, or a label under the control of Sox sequences or a sequence transactivatable by a SOX polypeptide, can be incorporated into a vector according to techniques known in the art. As used herein, vector (or plasmid) refers to discrete elements that are used to introduce heterologous DNA into cells for expression. Selection and use of such vehicles arc well within the skill of the artisan. The vector components generally include, but are not limited to, one or more of the following: an origin of replication, one or more marker genes, an enhancer element, a promoter, a transcription termination sequence and a signal sequence.

Most expression vectors are shuttle vectors, i.e. they are capable of replication in at least one class of organisms but can be transfected into another class of organisms for expression. For example, a vector is cloned in E. coli and then the same vector is transfected into mammalian cells even though it is not capable of replicating independently of the host cell chromosome. Advantageously, an expression and cloning vector may contain a selection gene, also referred to as selectable marker, other than that intended for marking Sox-expressing cells. This gene may encode a protein necessary for the survival or growth of transformed host cells grown in a selective culture medium. Host cells not transformed with the vector containing the selection gene will not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available from complex media.

Since the replication of vectors is conveniently done in E. coli, an E. coli genetic marker and an E. coli origin of replication are advantageously included. These can be obtained from E. coli plasmids, such as pBR322, Bluescript® vector or a pUC plasmid, e.g. pUC18 or pUC19, which contain both an E. coli replication origin and an E. coli genetic marker conferring resistance to antibiotics, such as ampicillin.

Expression vectors usually contain a promoter that is recognized by the host organism and is operably linked to a Sox gene, or a label-encoding, nucleic acid. Such a promoter may be inducible by factors which induce Sox gene expression, or by a SOX polypeptide itself. The promoters are operably linked to DNA encoding a SOX polypeptide by removing the promoter from the source DNA and inserting the isolated promoter sequence into the vector. Both the native Sox promoter sequences and many heterologous promoters may be used to direct amplification and/or expression of SOX DNA. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Control sequences, comprising a promoter and optionally enhancer(s), may be derived from the human or other Sox genes. Alternatively, any suitable promoter may be used, when placed under the control of a SOX-inducible element. In such a construct, the promoter selected should have a low residual level of activity (<10% of the activity observed in the presence of a SOX polypeptide), such as to minimize expression of the label in the absence of SOX polypeptide expression.

The vectors may also contain sequences necessary for the termination of transcription and for stabilizing the mRNA. Such sequences are commonly available from the 5′ and 3′ untranslated regions of eukaryotic or viral DNAs or cDNAs. These regions contain nucleotide segments transcribed as polyadenylated fragments in the untranslated portion of the mRNA encoding a SOX polypeptide or the label.

An expression vector includes any vector capable of expressing a SOX polypeptide or any marker or label encoding nucleic acid that is operatively linked to a regulatory sequence, such as promoter regions, that are capable of regulating expression of such DNAs. Thus, an expression vector refers to a recombinant DNA or RNA construct, such as a plasmid, a phage, recombinant virus or other vector, that upon introduction into an appropriate host cell, results in expression of the cloned DNA. Appropriate expression vectors are well known to those with ordinary skill in the art and include those that are replicable in eukaryotic and/or prokaryotic cells and those that remain episomal or those which integrate into the host cell genome. For example, DNAs encoding SOX1 may be inserted into a vector suitable for expression of cDNAs in mammalian cells e.g. a CMV enhancer-based vector such as pEVRF (Matthias et al., (1989) NAR 17, 6418).

Particularly useful for practicing the present invention are expression vectors that provide for the transient expression of DNA encoding a SOX polypeptide or a label in mammalian cells. Transient expression usually involves the use of an expression vector that is able to replicate efficiently in a host cell, such that the host cell accumulates many copies of the expression vector, and, in turn. synthesizes high levels of the SOX polypeptide or a label or marker. For the purposes of the present invention, transient expression systems are useful e.g. for identifying SOX expressing cells or for inducing a pluripotent cell to differentiate.

Construction of vectors according to the invention employs conventional techniques, for example as described in Sambrook et al., 1989. Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing gene expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis), or in situ hybridization, using an appropriately labeled probe which may be based on a sequence provided herein. Those skilled in the art will readily envisage how these methods may be modified, if desired.

Dosage and Mode of Administration:

By way of example, a patient in need of a cell that is committed to a particular developmental pathway or a stem cell as described herein can be treated as follows. Cells of the invention can be administered to the patient, preferably in a biologically compatible solution or a pharmaceutically acceptable delivery vehicle, by ingestion, injection, inhalation or any number of other methods. A preferred method is endoscopic retrograde injection. The dosages administered will vary from patient to patient; a “therapeutically effective dose” can be determined, for example but not limited to, by the level of enhancement of function. Monitoring levels of stem cell introduction, the level of expression of certain genes affected by such transfer, and/or the presence or levels of the encoded product will also enable one skilled in the art to select and adjust the dosages administered. Generally, a composition including a stem cell of the invention will be administered in a single dose in the range of 10⁵-10⁸ cells per kg body weight, preferably in the range of 10⁶-10⁷ cells per kg body weight. This dosage may be repeated daily, weekly, monthly, yearly, or as considered appropriate by the treating physician. The invention provides that cell populations can also be removed from the patient or otherwise provided, expanded ex vivo, transduced with a plasmid containing a therapeutic gene if desired, and then reintroduced into the patient.

Pharmaceutical Compositions:

The invention provides for compositions comprising a stem cell or a cell commited to a particular developmental pathway according to the invention admixed with a physiologically compatible carrier. As used herein, “physiologically compatible carrier” refers to a physiologically acceptable diluent such as water, phosphate buffered saline, or saline, and further may include an adjuvant. Adjuvants such as incomplete Freund's adjuvant, aluminum phosphate, aluminum hydroxide, or alum are materials well known in the art.

The invention also provides for pharmaceutical compositions. In addition to the active ingredients, these pharmaceutical compositions may contain suitable pharmaceutically acceptable carrier preparations which can be used pharmaceutically.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer' solution, or physiologically buffered saline. Aqueous injection suspensions may contain substances which increase the viscosity of the suspension, such as sodium carboxymethyl cellulose, sorbitol, or dextran. Additionally, suspensions of the active solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For nasal administration, penetrants appropriate to the particular barrier to be permeated are used in the formulation. Such penetrants are generally known in the art.

The pharmaceutical compositions of the present invention may be manufactured in a manner known in the art, e.g. by means of conventional mixing, dissolving, granulating, dragee-making, levitating, emulsifying, encapsulating, entrapping or lyophilizing processes.

The pharmaceutical composition may be provided as a salt and can be formed with many acids, including but not limited to hydrochloric, sulfuric, acetic, lactic, tartaric, malic, succinic, etc. . . . Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a Ph range of 4.5 to 5.5 that is combined with buffer prior to use.

After pharmaceutical compositions comprising a compound of the invention formulated in a acceptable carrier have been prepared, they can be placed in an appropriate container and labeled for treatment of an indicated condition with information including amount, frequency and method of administration.

Use

Cells obtained according to the invention may be employed in a number of ways. Of course, the expression of Sox genes has important implications for the study of embryonal differentiation; the generation and selection of specific cell lineages will provide material for basic research.

Moreover, the invention has medical and diagnostic applications. The detection of Sox expressing cells is important in clinical neurology and in diagnosing and treating cancers of the nervous system. Accordingly, the invention provides a method for detecting the presence of a neuroblast as described above for diagnostic purposes.

Stem cells are also useful for the treatment of disorders of any given tissue, particularly for the treatment of neurological disorders and especially for repair of accidentally induced trauma in the CNS or for the correction of congenital or pathological diseases of the CNS.

Moreover, in applications involving somatic gene therapy designed to correct a genetic defect, the removal, treatment and replacement of pluripotent cells which are actively dividing has clear advantages, providing a constant source of modified neural cells to permanently treat the targeted defect. Sox control sequences may be used specifically to direct transgene expression in specified cells where this is desired. Moreover, gene expression can be directed to terminally differentiated cell types derived from pluripotent cells by the use of other control sequences, such as NF-1 control sequences which direct expression of NF-1 in mature neurons in vivo.

A significant advantage of the methods described herein is that a patient in need of treatment can act as a self-donor. In other words, cells may be isolated from the patient and either sorted to extract desired cell types, or treated in order to differentiate the required cells as described, from specific or general precursors.

The above disclosure generally describes the present invention. A more complete understanding can be obtained by reference to the following specific examples, which are provided herein for purposes of illustration only and are not intended to limit the scope of the invention.

### EXAMPLES

Material and Methods

Manufacture of SOX1 Polyclonal Antibodies:

A 622bp HincII fragment encoding sequences C-terminal of the HMG box of SOX1 (207 a.a.) is fused in frame to the bacterial GST gene in the construct pGEX3X. Fusion protein is induced and purified as described by Smith a& Johnson (1988). Rabbits are treated with a course of injections as recommended by Smith & Johnson (1988): each injection contains 250 μg of fusion protein. Two final bleeds, FB43 and FB44 , are obtained from the rabbits prior to the preparation of polyclonal sera.

Immunocytochemistry:

Embryos, P19 cells and neural plate explants are examined using standard techniques (Placzek et al., 1993). Antibodies are used at the following dilutions: anti-SOX1 PAb (1:500); K2 anti-HNF3β MAb (1:40); 6G3 anti-FP3 MAb (1:10); anti-3A10 MAb (1:10); anti-2H3(Neurofilament-160) MAb (1:10); 4D5 anti-Islet1 MAb (1:1000); anti-SSEA1 MAb (1:80) (Hybridoma Bank); anti-NESTINE MAb (1:10) (Hybridoma Bank); anti-BrDU MAb (1:500) (Sigma). Appropriate secondary antibodies (TAGO and Sigma) are conjugated to fluorescein isothiocyanate (FITC), Cy2 or Cy3.

BrDU Analysis:

Pregnant mice are injected intraperitoneally with 50 μg/g of body weight of 5-bromo-2deoxyuridine (BrDU) (Sigma) in 0.9% NaCl and sacrificed two hours after injection. Embryos are fixed and sectioned as described above. The slides are washed twice in PBS, and incubated in 0.2% HC1 at 37° C. for 30 minutes, then rinsed thoroughly with PBS, followed by three rinses with PBS/0.1% Triton/1% heat inactivated goat serum (P-T-G). Monoclonal anti-BrDU (1:500 dilution in P-T-G) is applied to the sections and incubated at 4° C. overnight. Sequential sections are incubated in SOX1 antibody (1:500 dilution in P-T-G) at 4° C. overnight. The slides are washed twice in P-T-G, then incubated in the appropriate secondary antibody for 30 minutes at room temperature, washed with P-T-G and mounted.

P19 Cell Culture and Retinoic Acid Treatment:

P19 cells are cultured as previously described (Rudnichy & McBurney, 1987). To induce differentiation, cells are allowed to aggregate in bacterial grade petri dishes alone, in the presence of 1 μM retinoic acid or in the presence of 5 mM IPTG. In certain embodiments, cells are allowed to aggregate in the presence of both 1 μM retinoic acid and 5 mM IPTG. After 4 days of aggregation in the presence of inducing agents, cells are plated on tissue culture chamber slides. The cells are allowed to adhere and grow for 4-5 days, with media changes every 24 hours: For immunofluorescence, cells are grown on tissue culture chamber slides coated with 0.1% gelatin, washed once with PBS, fixed at room temperature in 1×MEMFA for 1 hour, washed in P-T-G twice; then stained with the appropriate antibody.

Cell Counting Analysis:

For cell counting experiments P19 transfectant cell lines are induced to differentiate, plated on gelatine coated slides, fixed at room temperature in 1×MEMFA for one hour at day 6-8 for neurons. Cells are stained with Neurofilament (2H3) antibody and photographed using an Olympus fluorescence microscope. Cell counts are expressed as percentages of total cells in a field. Eight fields from two different experiments are counted for each P19 clone.

Plasmids and Transfection:

To construct the SOX1 expression vector, pRSVopSox1, the POP113CAT operator vector (Stratagene) is digested with NotI, and end-filled with the Kpn/Stu (position 431-1694) fragment of the Sox1 cDNA. The P3′SS, eukaryotic Lac repressor expressing vector (obtained from Stratagene) is transfected into P19 cells by lipofection. Stable transformants are selected in 250 μg/ml of hygromycin. Expanded clones (250) are isolated and examined for expression of the Lac repressor by indirect immunofluorescence with anti-lac PAb (Stratagene). Four cell lines are isolated (P3′SS-10, 13, 22 and 47) which show ubiquitous and constitutive expression of the Lac repressor. P3′SS-10 is chosen for the subsequent experiments. P3′SS-10 is then transfected with pRSVopSox1 by lipofection. Stable clones are selected using 500 μg/ml G481. 250 clones are expanded and analyzed for inducible Sox1 expression by RNase protection and immunocytochemistry with SOX1 antibody.

RNase Protection Assays:

Total RNA is prepared from P19 cells and RNase protection assays are carried out using 5μg of P19 cell RAN as described by Capel et al., (1993). Anti-sense labeled probes are derived from the 396 bp SmaI-BspH1 fragment (position 1467-1863) of the Sox1 cDNA, a 215 bp Bsal exon 4 specific fragment of Wnt1 cDNA, a PvuII digest of the Mash1 cDNA (Johnson et al., 1992) and a NotI digest of SAP D cDNA is used as a loading control (Dresser et al., 1995).

RT-PCR:

Total RNA is prepared from P19 cells as described by Capel et al., (1993). Reserve transcription, and PCR reactions are performed as described by Okabe et al., (1996).

Rat Lateral Neural Plate Explants:

Lateral neural plates (LNP) are isolated from days 8.5-9.0 rat embryos from prospective hindbrain and spinal cord regions as previously described (Placzek et al., 1993). Notochord explants are dissected from HR stage 608 chick embryos as previously described (Placzek et al., 1993). Explants are embedded in collagen and cultured (Placzek et al., 1993) for 24, 48 and 96 hours. Purified rat SHH-N (Ericson et al., 1996) is added to cultures at concentrations within the effective ranges used in other assays (Ericson et al., 1996).

### EXAMPLE 1

SOX1 is Expressed During Early Neural Development

SOX1 expression during mouse and rat neurulation is analyzed using a rabbit polyclonal antibody against the SOX1 C-terminal region. In the mouse, expression of SOX1 is first detected at 7.5 days post coitum (dpc) in the anterior half of the late-streak egg cylinder. Cross-sections through the embryo at this stage reveal expression in columnar ectodermal cells, which appear to define the neural plate, while cells located more laterally are negative. Thus, SOX1 expression at this stage is specific to the neural plate. SOX1 is maintained in all neuroepitheial cells along the entire anteroposterior axis as the neural plate bends (8.0-8.5 dpc, as demonstrated by cross-sections of a 2 somite mouse embryos where Sox1 expression is limited to neural folds,) and fuses to form the neural tube (9.0-9.5 dpc, where Sox1 labeling is seen to be restricted to the neural tube in cross-sections of 10-12 somite mouse embryos) data not shown. The pattern of expression of SOX1 in the rat is similar to that in the mouse. The expression of SOX1 throughout the neural plate and early neural tube implies a similarity amongst these cells. After neural tube closure, neuroepithelial cells begin to differentiate into defined classes of neurons at specific dorsoventral (D/V) positions within the spinal cord (Altman & Bayer, 1984, Tanabe & Jessell, 1996). As development proceeds, Sox1 is downregulated in a stereotyped manner in cells along the D/V axis of the neural tube. In the spinal cord, expression is first downregulated in cells that occupy the ventral midline (cross-sections of the thoracic region of 20 somite mouse embryos reveal a lack of SOX1 staining in this area), then the ventral motor horns (corresponding lack of staining being visible in cross section of 30-35 somite embryos) and subsequently the dorsal regions. These regions appear to correlate with floor plate, motor neurons and sensory relay interneurons, respectively.

To ascertain this a series of antibody double-labeling experiments are performed in rat embryos. The SOX1 antibody is used in combination with a panel of antigenic markers which identify cells of the floor plate and mature neurons (Neurofilament (NF-1): labeled with contrasting color markers and visualized in an E11 rat embryo). Expression of SOX1 and expression of these markers is almost entirely mutually exclusive. In the ventral spinal cord or the 10.0-12.0 dpc mouse embryo, SOX1 expression is maintained only in ‘region X’ (Yamada et al., 1991), as revealed by immunolabeling of two streams of cells located between the differentiated floor plate and ventral motor horns in 30-35 somite embryos. Eventually, by 13.5 dpc, SOX1 expression is restricted to a thin ventricular zone in the CNS. SOX1 expression is not detected in the peripheral nervous system (PNS). These expression profiles suggest that SOX1 is expressed by early neural cells in the CNS and is downregulated in the developing neural tube coincident with neural differentiation.

### Example 2

SOX1 Marks Proliferating Cells within the Embryonic Neural Tube

The uniform expression of SOX1 in the neural plate and early neural tube followed by its downregulation along the D/V axis and restriction to the ventricular zone is reminiscent of the pattern of cell proliferation in the developing central nervous system (Sauer, 1935; Fujita, 1963; Altman & Bayer, 1984). In the neural plate and early neural tube, proliferating progenitor cells are organized in a pseudostratified epithelium in which the processes of these cells extend from the inner luminal to the outer mantle surface. At later stages the neural tube becomes progressively thicker and can be divided into different zones. The proliferating CNS progenitors are largely restricted to the inner ventricular zone (VZ) around the lumen. They begin to migrate away from the lumen while in S-phase, and after completing their final mitosis, migrate to the outer layer, the marginal zone (MZ). In the 10.5 dpc mouse embryo, SOX1 expression is detected, using an anti-SOX1 antibody, throughout the pseudostratified epithelium of the posterior neural tube and is restricted to the ventricular zone in the more mature anterior region of the neural tube. In order to evaluate the relationship between SOX1 expression and proliferating CNS cells, the cells are directly assayed for proliferation by monitoring the incorporation of bromodeoxyuridine (BrDU) with an anti-BrDU antibody. Pregnant mouse females at 10.5 dpc are injected with BrDU two hours prior to dissection to detect proliferating cells. Embryos are then fixed, sectioned and double-labeled for BrDU incorporation and SOX1 expression. Similar to SOX1 expressing cells, those that incorporate BrDU are found throughout the posterior neural tube in 10.5 dpc mouse embryos and lie in the ventricular zone of the anterior neural tube. All cells that incorporate BrDU also express SOX1. SOX1-positive cells that do not incorporate BrDU are restricted to the luminar surface of the ventricular zone. In contrast, no SOX1 nor BrDU-positive cells are detected in the outer marginal zone. These results show that SOX1 is expressed in dividing neuroepithelial cells within the embryonic CNS.

### Example 3

SOX1 is Downregulated in most Commitfed Cells

The mutual exclusion of SOX1 and markers of committed differentiated cells such as Islet1 (Pfaff et al., 1996) raises the possibility that the downregulation of SOX1 may be a prerequisite step for the differentiation in neural plate explants in vitro. Isolated neural plates explants are cultured with known inducers of ventral neural cells, namely the notochord and purified Sonic Hedgehog protein. The expression of SOX1 and incorporation of BrDU is then compared to the expression of three markers of ventral cells, Islet1, FP3 and HNF3β. Consistent with our observations in vivo both the expression of SOX1 and Islet1 as well as SOX1 and FP3 is mutually exclusive in neural plate explants cultured adjacent to notochord (n=8) or in the presence of purified Sonic Hedgehog protein as seen in E9 rat neural plate tissue cultured with Sonic Hedgehog protein for 48 hours and stained with anti-SOX1 and anti-Islet1 antibodies. Similarly, the incorporation of both BrDU and Islet1 as well as BrDU and FP3 (detected using an antiFP3 antibody) is mutually exclusive, in contrast, the domain of expression of HNF3β is found to extend beyond that of FP3 and into the region of BrDU positive cells.

To determine whether a similar population of cells could be detected in vivo, embryos are analyzed for co-expression of FP3 and HNF3β and for co-expression of BrDU and HNF3β. We find that medial floor plate cells co-express HNF3β and FP3 but do not incorporate BrDU, whereas lateral floor plate cells express only HNF3β and incorporate BrDU. HNF3β thus provides a marker for cells that are mitotically active but have begun to differentiate.

These cells, occupying the medial regions of the floor plate, express HNF3β but not SOX1. In contrast cells occupying lateral regions of the floor plate co-express HNF3β and SOX1. These observations, together with the mutually exclusive expression of SOX1 with Islet1 and FP3 in ventral neural cells provide evidence that SOX1 is downregulated as cells exit mitosis and not at the onset of cell differentiation.

### Example 4

SOX1 Expression is Associated with Neural Differentiation

Neural induction is accompanied by the onset of new gene expression which in turn enables the formation of neural rather than epidermal tissue. The early and apparently uniform expression of SOX1 in neural cells, together with observations that Sox genes may affect cell lineage decisions (see Introduction), raises the possibility that SOX1 expression is an early response to neural inducing signals and that its expression may be involved in directing cells towards a neural fate. To address whether SOX1 plays a role in establishing neural fate a P19 cell culture system is used as an in vitro model system in which to analyze SOX1 expression and the effects of its misexpression.

P19 cells are an embryonal carcinoma cell line with the ability to differentiate into all three germ layers (McBurney, 1993). In the undifferentiated state P19 cells morphologically resemble an uncommitted primitive ectodermal cell and express the cell surface antigen SSEA-1. These cells have a very low rate of spontaneous differentiation when grown in a monolayer in the absence of chemical inducers. P19 cells grown as aggregates, however, differentiate partially into endodermal cells. Furthermore, with the addition of retinoic acid, aggregated P19 cells differentiate into neuroepithelial-like cells (Jone-Villeneuve et al., 1982). These express neuroepithelial markers such as NCAM, intermediate filament NESTIN, MASH1 (Johnson et al., 1992) and WNT1 (St. Arnaud et al., 1989). When plated onto a substrate, about 15% of these cells differentiate into mature neurons expressing Neurofilament. Thus, in this in vitro model system retinoic acid acts as a “neural inducer”. Initially, the expression of Sox1 in P19 cells is examined by both RNase protection and immunocytochemistry. The features of Sox1 expression in P19 cells are similar to those observed in prospective neural tissue in vivo. Sox1 mRNA and protein cannot be detected in undifferentiated P19 cells which express the cell-surface antigen SSEA1 when analyzed using anti-SOX1 and anti-SSEA antibodies, and by RNase protection. Similarly, when P19 cells are differentiated as aggregates without the addition of chemical inducers, SOX1 is not expressed as determined by RNase protection. In contrast, SOX1 is rapidly induced during neural differentiation when aggregated P19 cells are differentiated in the presence of retinoic acid. Sox1 thus behaves similarly to other neuroepithelial markers such as Mash1 and Wnt1, the transcripts of which are detected in retinoic acid-treated P19 cells by RNase protection.

When retinoic acid-treated P19 cell aggregates are plated onto tissue culture substrate, about 15% of the cells differentiate into mature process-bearing, Neurofilament-expressing neurons. Double-label immunofluorescence is used to simultaneously detect SOX1 and Neurofilament, to examine the expression of SOX1 in P19 cells displaying a fully differentiated neuronal morphology. SOX1 immunoreactivity is not detected in process-bearing Neurofilament-positive neurons. Thus, as in vivo, SOX1 is expressed by P19 cells when they first assume a neural fate but it is then downregulated with their terminal differentiation.

### Example 5

Use of SOX1 to Direct Cells to a Neural Fate

The previous data suggest that in P19 cells, as in vivo, SOX1 expression is induced at a time when neuroepithelial cells begin to differentiate. If SOX1 plays a role in directing cells towards the neural fate, expression of SOX1 in P19 cells may be able to substitute for retinoic acid to initiate neural differentiation. Endogenous SOX1 is accordingly activated in P19 cells using an inducible eukaryotic lac repressor-operator expression system. To establish this system a clonal line of P19 cells is generated which constitutively and ubiquitously expresses the lac repressor. This parent line (P3′SS-10) is transfected with pRSVopSox1, a vector containing the Sox1 cDNA under the regulation of an inducible RSV promoter and stable lines are established. In the uninduced state, without the addition of isopropyl-β-d-thiogalactase (IPTG) these lines express high levels of the lac repressor that binds to operon sites upstream of the RSV promoter and thus blocks transcription of Sox1. Upon addition of IPTG a conformational change occurs, decreasing the affinity of the repressor and resulting in the activation of pRSVopSox1. Approximately 250 clones of transfectants are isolated in the repressed state. Using RNase protection and immunocytochemistry assays three clones are selected (708-13, 708-16 and 708-21) that express high levels of RSVopSox1 in response to IPTG.

The pluripotentiality of these clones is not compromised by the transfection and selection. All three lines express SSEA1 in the uninduced state. Furthermore, when aggregated in retinoic acid the uninduced clones initiate expression of endogenous Sox1 and differentiate into mature Neurofilament-expressing neurons after plating, in a manner similar to wild-type P19 untransfected cells.

In order to address whether expression of SOX1 can initiate neural differentiation and thereby substitute for the requirement of retinoic acid, it is determined whether the transient exposure of P19 aggregates to retinoic acid can be replaced by a transient induction of RSVopSox1, through the addition of IPTG. Wild-type P19 cells and transfected P19 clones (708-13, 708-16 and 708-21) are cultured as aggregates for 96 hours with or without the addition of IPTG. After 96 hours RNA is isolated from half of the aggregates for RNase protection and/or RT-PCR assays. The remaining aggregates are plated onto tissue culture substrate, allowed to differentiate for three days without further addition of IPTG and then scored for the expression of a panel of neuroepithelial and neuronal markers by immunocytochemistry. These conditions are the same as those used for retinoic acid-induced differentiation of wild-type P19 cells. After 96 hours the clones induced to express RSVopSox1 with IPTG express endogenous Sox1 and Mash1. The expression of these two neuroepithelial markers is similar to that seen in wild-type cells induced with retinoic acid. In addition the IPTG induced clones expressed NESTIN and Hoxa7 (Mahn et al., 1988). Further differentiation of the transiently-induced clones on the tissue culture substrate showed the presence of mature neurons as demonstrated by Neurofilament-positive, 3A10-positive and Islet1-positive cells. All three clones 708-13, 708-16 and 708-21 differentiate in this manner although the number of mature neurons produced is variable. The number of differentiated neurons formed in the IPTG induced clones is estimated by determining the number of Neurofilament-positive cells in a given field of cells. The number of neurons ranges from 6-8% for clone 708-13, 15-20% for clone 708-16 and 20-25% for clone 708-21. The latter two clones show uniform and ubiquitous induction of SOX1 expression whereas expression in clone 708-13 is not in all cells (data not shown). In addition, the transiently induced clones generate GFAP-positive cells indicating glial cell differentiation. None of these markers is detected in wild-type P19 cells cultured in the presence of IPTG or in clones 708-13, 708-16, and 708-21 cultured in the absence of IPTG. The expression of SOX1, both in vivo and in vitro, is mutually exclusive with mature neuronal markers such as Neurofilament and Islet1. To examine SOX1 expression in the mature neurons generated in the transiently-induced clones, double-label immunofluorescence is used to simultaneously detect SOX1 and Neurofilament. No SOX1 expression could be detected in cells positive for Neurofilament in these cultures (data not shown).

### Example 6

Use of SOX2 to Isolate Neural Precursors

Like SOX1, SOX2 is expressed in a pan-neural fashion from mid-streak stages on during mouse embryogenesis. However, at the beginning of gastrulation the initial phase of SOX2 and SOX3 expression is pan-ectodermal along the entire proximal/distal axis of the egg cylinder. In light of the Xenopus data which proposes that “neural” is “default” for early gastrula ectoderm, an intriguing possibility is that SOX2 expression may reflect the potential of the mouse primitive ectoderm to be neural. It has been demonstrated that the Xenopus Sox2 can synergize with FGF signaling to initiate neural differentiation, indicating a role for SOX2 in neural specification. In addition, Drosophila Dichaete mutants (Dichaete being the Drosophila orthologue of vertebrate SOX2) display defects in the specification and differentiation of midline neural cells which can be rescued by mouse SOX2. Moreover, we have demonstrated using clonal cultures of embryonic neural tubes that SOX2 is expressed in the multipotent proliferating neural stem cells. Thus SOX2 serves as good tool by which to isolate neuroepithelial cells both from embryonic neural tissue and embryonic stem cells. The following is a detailed example of the isolation of neural epithelial cells from embryonic stem cells by SOX selection.

For induction of neural differentiation, ES cells are aggregated in suspension to form embryoid bodies, exposed to retinoic acid, and then allowed to reattach to a substratum. Neuronal-like cells can be detected in the out-growths, accompanied by a variety of other cell types. Two variations are introduced to the protocol that enhances the final representation of neuronal cells. First, the embryoid bodies are dissociated before plating. This results in a homogeneous dispersion and terminates inductive and selective effects within the embryoid bodies. Second, cells are plated in a defined culture medium—DMEM/F12 plus N2 supplement—on substrata coated with poly-D-lysine and laminin, which support attachment and outgrowth of neuronal cells.

These procedures have an additive effect on the proportion of neural cells in the cultures. When combined, up to 50% of viable cells extended neuritic processes and become immunoreactive for the neuronal markers neurofilament light and heavy chains, microtubule-associated proteins, MAP2 and tau, or β-tubulin III.

Immunostaining of freshly plated cells with antibodies against Sox1 and Sox2 reveals that 40-50% of the cells are positive for each marker. This approximates to the final proportion of differentiated neural cells, consistent with the notion that cells expressing Sox1 and Sox2 correspond to neural-restricted progenitors.

To attempt to isolate the neural progenitor pool, ES cells are used in which the bifunctional selection marker/reporter gene βgeo has been integrated into the Sox2 gene by homologous recombination. When induced to differentiate as described above, approximately 50% of these cells stain for β-galactosidase activity, consistent with the proportion of cells that express Sox2 protein. Therefore, application of G418 to the differentiating cultures should eliminate Sox2-negative non-neural cells. G418 (200 g/ml) is added after retinoic-acid induction, either during embryoid body culture or upon plating. In both conditions appreciable cell killing is evident. Crucially, however, large numbers of cells survive that exhibit the small, ovoid morphology typical of neuroepithelial cells. Over 90% of these cells show prominent P-galactosidase staining. Expression of Sox1 and Sox2 proteins is confirmed by immunostaining. Consistent with a neuroepithelial identity, the cells also express nestin.

Accordingly, neural cell types may be isolated by expression of a marker associated with Sox2, starting with a population of totipotent cells which has been induced to differentiate inter alia into a neural pathway.

In order to determine whether the Sox2-selected population have proliferative capacity, βFGF is added to plated cultures. This results in a major stimulation of cell division. The expanded cells predominantly retain undifferentiated neural morphology and show strong X-gal staining indicative of Sox2 expression. Such cultures can be amplified and serially passaged for at least three weeks, which is significantly longer than the proliferative phase of neurogenesis in the mouse embryo.

In the absence of mitogen, Sox2-selected precursor cells begin to extend neuritic processes within 48 hours and by 96 hours form a network of neuron-like cells. The pan-neuronal markers neurofilament light chain, microtubule-associated proteins, MAP2 and tau, and β-tubulin III are detectable from 48 hours onwards, coincident with down-regulation of Sox2 expression. By 96 hours, over 90% of cells express neuronal markers, including neurofilament heavy chain and synapsin I. Cells of non-neuronal morphology are rarely apparent, with the exception of the occasional GFAP-positive astrocyte. Astrocyte numbers increase if serum of FGF is added to the cultures. Maturation of the neuronal cells, evidenced by production of gamma-aminobutyric acid (GABA) and glutamate neurotransmitters, and further elongation of neurites with dendritic sprouting is achieved on transfer to Neurobasal medium supplemented with B27 and horse serum.

This ability to generate pure populations of neural epithelial cells, combined with the relative ease of genetic modification of ES cells, offers a new route for manipulation and characterization of neuronal development and cell biology. The finding that major cellular components of embryoid bodies can be ablated without apparently perturbing development of the surviving cells also indicates that this strategy can be adapted to isolate stem or precursor cells for other lineages. An important attribute is that unlike immunopurification techniques this approach is not limited to cell-surface antigens but can be applied to any Sox gene. Selected populations can readily be refined by introducing independent markers into more than one gene.

The advantage of targeting progenitors as opposed to differentiated cells is the potential for subsequent amplification and directed differentiation both in vitro and in vivo. ES cell derivatives can colonize host tissue and differentiate after transplantation into adult recipients. Grafts of whole embryoid body cultures, however, also give rise to teratomas and other benign or malignant growths. Furthermore, heterologous cells may interfere with trophic signals and guidance cues from host tissue to transplanted cells. Prior lineage purification should eliminate these problems and enable the multipotentiality of ES cells to be harnessed effectively for application in cellular transplantation.

Other Embodiments

Other Embodiments are within the claims that follow.

1. A method for isolating a pluripotent cell which is at least partially committed to a given developmental pathway comprising the steps of:
(a) selecting a population of pluripotent cells;
(b) sorting the cells according to Sox gene expression; and
(c) isolating those cells which express a given Sox gene.

2. A method according to claim 1, wherein the population of cells for is derived from CNS tissue.

3. A method according to claim 1, wherein the population of cells is derived from a cell culture.

4. A method according to any preceding claim, wherein the expression of the Sox gene is detected by nucleic acid hybridization.

5. A method according to any one of claims 1 up to 3, wherein the expression of the Sox gene is detected by a binding of a SOX polypeptide to a detectable ligand.

6. A method according to claim 5, wherein the detectable ligand is a labeled immunoglobulin.

7. A method according to claim 5, wherein the detectable ligand is a labeled oligonucleotide complementary to Sox mRNA.

8. A method according to any preceding claim, wherein the expression of the Sox gene is detected by FACS analysis.

9. A method for isolating a desired cell type from a population of cells, comprising the steps of:
(a) transfecting the population of cells with a genetic construct comprising a coding sequence encoding a detectable marker operatively linked to control regions sensitive to modulation by a SOX polypeptide;
(b) detecting the cells which express the selectable marker; and
(c) sorting the cells which express the selectable marker from the population of cells.

10. A method for isolating a neuroblastic cell from a population of cells, comprising the steps of:
(a) transfecting the population of cells with a genetic construct comprising a coding sequence encoding a detectable marker operatively linked to a control sequence which is transactivatable by a SOX polypeptide;
(b) detecting the cells which express the selectable marker; and
(c) sorting the cells which express the selectable marker from the population of cells.

11. A method according to claim 9 or claim 10, wherein the selectable marker is a fluorescent or luminescent polypeptide.

12. A method according to claim 9 or claim 10, wherein the selectable marker is a polypeptide detectable at the surface of the cell.

13. A method for producing a cell committed to a specified lineage, comprising the steps of:
(a) transfecting a pluripotent stem cell with a genetic construct comprising a coding sequence expressing a SOX polypeptide;
(b) culturing the stem cells in order to differentiate them into neural cells; and
(c) isolating the neural cells thereby produced.

14. A method according to claim 15, wherein the Sox sequence is operatively linked to an inducible promoter.

15. A method according to claim 13 or claim 14, wherein the cell is further transfected with a vector comprising a sequence encoding a regulator which modulates the expression of the Sox sequence.

16. A method according to any preceding claim, wherein the Sox gene is a member of Sox Group A.

17. A method according to claim 16, wherein the Sox gene is Sox1 or Sox2.
